(19) United States
(12) Patent Application Publication
(10) Pub. No.: US 2023/0055393 A1
(43) Pub. Date: Feb. 23, 2023

(54) PROTECTED STERILE INJECTOR WITH READY ISOTONIC

(71) Applicant: Atatürk Üniversitesi Bilimsel Arastirma Projeleri Birimi, Erzurum (TR)

(72) Inventors: Gürkan ÖZTÜRK, Erzurum (TR); Zühal YETIS DEMIR, Erzurum (TR); Necip ALTUNDAS, Erzurum (TR); Salih KARA, Erzurum (TR)

(21) Appl. No.: 17/760,164

(22) PCT Filed: Sep. 21, 2021
(86) PCT No.: PCT/TR2021/050968

§ 371 Date: Aug. 4, 2022

(30) Foreign Application Priority Data
Sep. 17, 2020 (TR) 2020/14821

(51) Int. Cl. A61M 5/315

(52) U.S. Cl. CPC A61M 5/31505; A61M 2005/3109

(57) ABSTRACT

A protected sterile injector with ready isotonic sodium chloride which provides drug dilution and administration in patient treatment services, contains isotonic sodium chloride liquid, includes at least one chamber having a hole for the medicament outlet, a piston connected to the chamber, ajutage that is located on the upper part of the chamber hole, and which is the part that allows for attachable-detachable use and is suitable for the use of different needle tips, a gasket which is connected to the upper part of the piston in the chamber, separating the liquid in the chamber and the empty space, at least one needle tip located in the outermost part of the chamber, a clamp connected to the outer part of the piston that prevents the involuntary outflow of the liquid in the chamber by preventing the movement of the piston and ensuring the controlled use of the liquid.

### FIELD OF THE INVENTION

The present invention relates to a ready-to-use protected sterile injector with isotonic sodium chloride solution that provides drug dilution and administration in patient treatment services used in the health sector.

The invention particularly relates to a protected sterile injector that provides ease of dilution of powder drugs and preparation of drugs in aseptic methods, is a drug diluting device that contains a plug-in needle tip of appropriate thickness and a reservoir with isotonic sodium chloride solution in different sizes and allows administering diluted drugs in appropriate doses directly into the vein or directly through the vascular access catheter.

### Present State of the Art

In current applications, single-use 100 cc isotonic sodium chloride solution solutions (serum), which are currently used in hospitals, are used to dilute drugs. An further injector is opened so as to dilute the drug, an antiseptic solution and sterile gauze are used to wipe the needle insertion area of the flexible bag (mediflex) containing isotonic sodium chloride solution. Since the needle tips are 22 G thick, there are difficulties in application. At the same time, the amount of isotonic sodium chloride liquid to be used to dilute the drug can be 20 cc, 50 cc and the remaining solution (serum) is wasted. These applications prevent the efficient use of time, waste materials, and cause difficulties in compliance with aseptic methods. Also, the use of unprotected needle tips causes stab wounds.

The following application is encountered in the literature in terms of the subject matter.

The patent application numbered TR 2019/14412 titled “Ready to Use Sodium Chloride Syringe” relates to a syringe that contains 0.9% sodium chloride solution, to maintain patency and reduce the risk of contamination, is made specifically of luer lock polypropylene for flushing vascular access devices. Said product is a product developed for the purpose of washing only the catheters where vascular access is provided. There is a head part suitable for the entry part of the direct catheter. There is no needle tip and it is not used to dilute the drug and administer the drug.

As a result, due to the abovementioned disadvantages and the insufficiency of the current solutions regarding the subject matter, a development is required to be made in the relevant technical field.

### BRIEF DESCRIPTION OF THE INVENTION

The present invention relates to protected sterile syringe with ready isotonic sodium chloride solution which fulfills the abovementioned requirements, eliminates all disadvantages and brings some additional advantages.

The main aim of the invention is to provide a protected sterile injector that will ease of dilution of powder drugs and preparation of drugs in aseptic methods by saving both material and time, is a drug diluting device that contains a plug-in needle tip of appropriate thickness and a reservoir with isotonic sodium chloride solution in different sizes and allows administering diluted drugs in appropriate doses directly into the vein or directly through the vascular access catheter.

An aim of the invention is to provide an economical injector with a chamber containing isotonic sodium chloride solution liquid in the amount to be used in the process, preventing material waste. Material waste is eliminated avoided by the chamber structure containing 0.9% NaCl solution liquid in different sizes as 5 ml-10 ml-20 ml.

An aim of the invention is to provide an injector that is suitable for direct administration of diluted drugs through the intravenous line, by means of the plug-in needle tip. The ajutage of the injector is suitable for use of all needle tips and is also suitable for use in drug administration to be made directly into the vein.

Another aim of the invention is to provide an injector with a protected needle tip mechanism that prevents stab wounds.

Another aim of the invention is to provide an injector with a 14 G-thick needle tip, which is the part that provides ease of process in the drug dilution.

Another aim of the invention is to provide an injector that ensures both time and cost advantages because it will be used with a single material instead of many materials.

Another aim of the invention is to provide a single use and sterilized packaged injector suitable for aseptic method.

In order to fulfill the above mentioned aim, the present invention relates to a protected sterile injector with ready isotonic sodium chloride which provides drug dilution and administration in patient treatment services, contains isotonic sodium chloride liquid, includes at least one chamber having a hole for the medicament outlet, a piston connected to the chamber used to deliver the drug therein by providing the movement of the liquid inside the chamber, ajutage that is located on the upper part of the chamber hole, is the part that allows for attachable-detachable use and is suitable for the use of different needle tips, gasket, which is connected to the upper part of the piston in the chamber, separating the liquid in the chamber and the empty space, at least one needle tip located in the outermost part of the chamber that provides the transfer of isotonic sodium chloride liquid to the drug, characterized by comprising; a clamp connected to the outer part of the piston that prevents the involuntary outflow of the liquid in the chamber by preventing the movement of the piston and ensuring the controlled use of the liquid.

The structural and characteristic features and all advantages of the present invention will be understood clearly by the following drawings and the detailed description made with reference to these drawings and therefore the evaluation shall be made by taking these figures and the detailed description into consideration.

### FIGURES CLARIFYING THE INVENTION

FIG. 1 is a perspective view of the inventive injector.

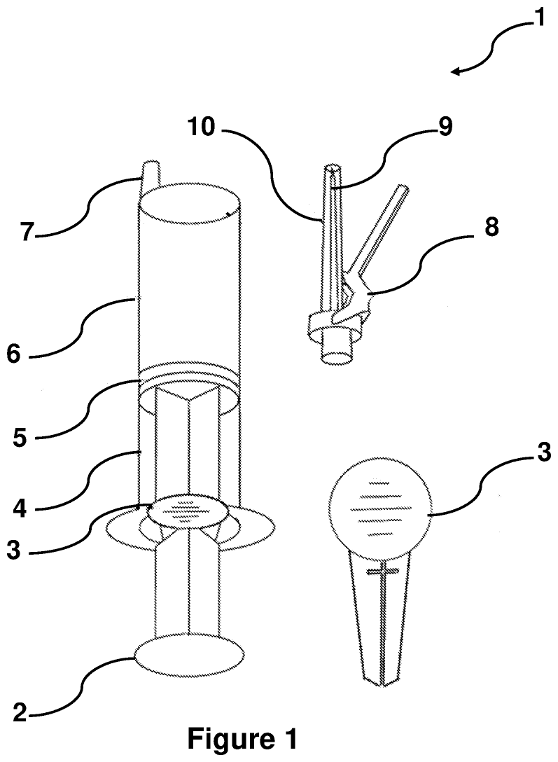

The figures are not required to be scaled and the details which are not necessary for understanding the present invention may be neglected. Moreover, the elements that are at least substantially identical or have at least substantially identical functions are shown by the same number.

### DESCRIPTION OF THE REFERENCES

1. Injector
2. Piston
3. Clamp
4. Empty space
5. Gasket
6. Chamber
7. Ajutage
8. Nail protection mechanism
9. Needle tip
10. Cap

### DETAILED DESCRIPTION OF THE INVENTION

In this detailed description, the inventive protected sterile injector with ready isotonic sodium chloride solution (1) is described only for clarifying the subject matter in a manner such that no limiting effect is created.

It relates to a protected sterile injector with ready isotonic sodium chloride (1) which provides drug dilution and administration in patient treatment services, contains isotonic sodium chloride liquid, includes at least one chamber (6) having a hole for the medicament outlet, a piston (2) connected to the chamber (6) used to deliver the drug therein by providing the movement of the liquid inside the chamber (6), ajutage (7) that is located on the upper part of the chamber (6) hole, is the part that allows for attachable-detachable use and is suitable for the use of different needle tips, gasket (5), which is connected to the upper part of the piston (2) in the chamber (6), separating the liquid in the chamber (6) and the empty space (4), at least one needle tip (9) located in the outermost part of the chamber that provides the transfer of isotonic sodium chloride liquid to the drug, clamp (3) connected to the outer part of the piston (2) that prevents the involuntary outflow of the liquid in the chamber (6) by preventing the movement of the piston (2) and ensuring the controlled use of the liquid.

The injector (1) comprises the following elements; chamber (6) which can be produced in different sizes as 5 ml-10 ml-20 ml, containing isotonic sodium chloride liquid, a nail protection mechanism (8) which is connected to the needle tip (9), which is the part that will close the needle tip (9) after use so as to prevent stab wounds, 14 G-thick needle tip (9), which is the part that will provide ease of application in transferring isotonic sodium chloride liquid to the drug and diluting the drug and cap (10) that will keep the needle tip (9) closed until use.

The inventive injector (1) is a packaged product. After the package is opened, the cap (10) of the needle tip is opened and inserted into the drug vial to be diluted, the clamp (3) is completely removed and the piston (2) is pushed into the solution vial. In case the drug dissolved in the vial is suitable for administration with the injector (1), the drug is withdrawn into the syringe (1) by pulling the piston (2) back again. After the needle tip (9) is removed from the vial, the cap (10) with the nail protection mechanism (8) is closed. If the drug is to be administered through the vascular access catheter, the needle tip (9) is separated from the ajutage (7) and the ajutage (7) is connected to the vascular access catheter, and the drug is administered into the vein by pushing the piston (2) at the appropriate speed, if the drug is to be administered directly to the vein, a needle of appropriate thickness (9) is inserted and applied directly to the vein. The nail protection mechanism (8) connected to the needle tip (9) can be pulled out of the ajutage (7) after the protected tip is closed at the end of the process.

Isotonic sodium chloride solution is an intravenous solution containing sodium and chloride ions, which are the basic building blocks of the body. It is prepared by dissolving sodium chloride (often called salt”) which a chemical substance is found in the blood, in water. Isotonic sodium chloride solution is used in the treatment of isotonic extracellular dehydration, in the treatment of sodium losses and in parenteral applications as a diluent solution of compatible drugs. During use as a drug diluent, the dose should be determined by the nature of the drug being diluted and the dosage schedule. In general, liquid between 50-250 ml is sufficient. The frequency of administration and dose are adjusted by the physician according to the clinical condition of the patient. The infusion rate of the recommended isotonic sodium chloride solution when used as a diluent is adjusted according to the recommended dose of the diluted drug. A solution containing 0.9% NaCl is used in said developed injector (1).

Specifically, the injector (1), which is a specific drug dilution tool that will save both material and time, in the dilution process of powder drugs, and facilitate drug preparation in aseptic methods can also be used in the administration of drugs that will be diluted and administered directly to the vein and administered directly to the vein through vascular catheters (such as branule, central vascular access catheter). The needle tip (9) of the protected sterile injector with ready isotonic sodium chloride solution (1) to be made is 14 G thick, and has an attachable-detachable property and it is planned in different sizes such as 5 cc-10 cc-20 cc.

The product is available in a single package, ready to use according to aseptic method and is disposable.

1. A protected sterile injector with ready isotonic sodium chloride which provides drug dilution and administration in patient treatment services, contains isotonic sodium chloride liquid, includes at least one chamber having a hole for a medicament outlet, a piston connected to the chamber used to deliver the drug therein by providing the movement of the liquid inside the chamber, ajutage that is located on the upper part of the chamber hole, is the part that allows for attachable-detachable use and is suitable for the use of different needle tips, a gasket, which is connected to the upper part of the piston in the chamber, separating the liquid in the chamber and the empty space, at least one needle tip located in the outermost part of the chamber that provides the transfer of isotonic sodium chloride liquid to the drug, wherein the injector comprises a clamp connected to the outer part of the piston that prevents the involuntary outflow of the liquid in the chamber preventing the movement of the piston and ensuring the controlled use of the liquid.

2. A ready-to-use protected sterile injector with isotonic sodium chloride solution according to claim 1, wherein the a needle tip has a thickness of at least 14 G, which provides ease of operation, and is connected to the ajutage.

3. A ready-to-use protected sterile injector with isotonic sodium chloride solution according to claim 1, comprising a nail protection mechanism which is connected to the needle tip, is the part that will close the needle tip after use so as to prevent stab wounds.

4. A ready-to-use protected sterile injector with isotonic sodium chloride solution according to claim 1, comprising a cap that will keep the needle tip closed until use.
